(19) United States
(12) Patent Application Publication
(10) Pub. No.: US 2024/0343759 A1
(43) Pub. Date: Oct. 17, 2024

(54) METHOD OF PREPARING PROCOLLAGEN FROM FRESHWATER FISH

(71) Applicants: Fu Yu Hsieh, Taipei (TW); Elizabeth Tien-Yang Cheng, Stowe, VT (US); Michael Furst, Stowe, VT (US)

(72) Inventors: Fu Yu Hsieh, Taipei (TW); Elizabeth Tien-Yang Cheng, Stowe, VT (US); Michael Furst, Stowe, VT (US)

(21) Appl. No.: 18/135,116

(22) Filed: Apr. 14, 2023

(51) Int. Cl. C07K 1/36; C07K 14/78

(52) U.S. Cl. CPC C07K 1/36; C07K 14/78

(57) ABSTRACT

The present invention discloses a method of preparing procollagen from freshwater fish. The method comprises the following steps: pressure treatment, physical crushing, mixing and emulsification, homogenization, refrigeration, cleaning, extraction, homogenization, inactivation, homogenization, and filtration. In the mixing and emulsification step, freshwater fish tissue containing collagen is mixed with a surfactant to remove endotoxins to below 0.25 EU/ml. The procollagen is extracted following the step of adding enzymes and an acid solution of pH 3 to 6 to the pretreated tissue. The pretreatment of freshwater fish tissue in this invention can effectively reduce the endotoxins to below 0.25 EU/ml. The extraction technique of this invention can extract and retain greater amounts of procollagen while increasing its denaturation temperature and preserving the intact, tightly twisted triple-helix bonds of type I procollagen.

### FIELD OF THE INVENTION

This invention relates to the preparation of procollagen, particularly to a method of removing pigments and endotoxins from freshwater fish tissue rich in collagen and extracting procollagen to meet the standards for medical device.

### COPYRIGHT NOTICE

A portion of the disclosure of this patent document contains material which is subject to copyright protection. The copyright owner has no objection to the facsimile reproduction by anyone of the patent document or the patent disclosure, as it appears in Patent and Trademark Office patent file or records, but reserves all copyright rights whatsoever. 37 CFR 1.71(d).

### STATEMENT REGARDING FEDERALLY FUNDED RESEARCH

This invention was not made under contract with an agency of the US Government, nor by any agency of the US Government.

### BACKGROUND OF THE INVENTION

21 Animal skin is comprised of 20% to 30% collagen extracellular matrix (ECM). Exposure to ultraviolet (UV) radiation and environmental pollution will cause loss of collagen in the skin, resulting in skin damage, roughness, and aging. In addition, many molecules such as the MMP enzyme family (matrix metallopeptidases or matrixins) can degrade the collagen ECM, making the skin structure unstable and causing skin aging.

Most of the collagen used in food, cosmetics, skincare, or pharmaceutical products on the market is collagen fragments. The loose, short-chain structure of collagen fragments makes them easily susceptible to decomposition by MMP enzymes. Moreover, due to their incomplete structure, collagen fragments cannot fully induce genetic responses of the collagen ECM. They can only provide modest collagen supplements for aging skin, making it difficult to activate the body's ability to repair aging skin.

Type I procollagen has been found to protect and repair skin tissues damaged by UV radiation effectively. In the skin, collagen fibrils are formed from the tightly twisted triple helical structure of type I procollagen, promote the proliferation of fibroblasts, and induce the secretion of growth factors to form the collagen ECM, protecting the skin from light damage.

The tightness and content of type I procollagen are closely related to the age of the skin. The collagen fibrils constructed by type I procollagen in mature skin are looser and shorter in chain length, resulting in a decrease in the number of fibroblasts and instability of the collagen ECM. Additionally, loose collagen fibrils are easily broken down into collagen fragments by MMP enzymes, leading to a reduced or lost ability to protect the skin. On the other hand, procollagen in the human body can attract fibroblasts to attach and grow, and secrete growth factors to promote the formation of ECM, forming skin protection. Moreover, the unique structure of procollagen can induce and actively initiate the UV signaling pathway for anti-UV repair.

In addition, the intact and tightly twisted triple helical structure of type I procollagen makes it less susceptible to external environmental damage and more suitable than collagen fragments normally for use in skincare, medical-grade cosmetic, and medical products. However, with current prior art technology, type I procollagen can only be synthesized, cannot be effectively extracted from animal tissues, and is a difficult material for mass production industrially, leading to a high market price.

Another problem is that of endotoxins. Endotoxins contamination is common in collagen materials. This is caused by the gram-negative bacteria, which are commonly found in animals' habitats and can adhere to the skin or be absorbed into the body of animals, and when in the process of extracting procollagen, they will die and release lipopolysaccharides (LPS, also known as endotoxins). Endotoxins can be dangerous when they enter the bloodstream, causing microcirculation disorders, septic shock, disseminated intravascular coagulation (DIC), and fever. In addition, the human body is extremely sensitive to the pyrogenic effects of endotoxins. Even tiny amounts (1-5 ng/kg body weight) of endotoxins injected into the body can cause fever and other harm.

Endotoxin is a non-protein component with a stable structure that must be heated at 250° C. for 2 to 4 hours to destroy its activity. Unfortunately, currently available collagen on the market has a denaturation temperature below 100° C., which makes it difficult to remove endotoxins using the 250° C. heating method. Additionally, soaking collagen in alcohol and acetone can remove endotoxins, but this method often results in protein denaturation. Some literature suggests that endotoxins can be removed by using Triton X-114 cloud point extraction, but this method can reduce the endotoxins only from 10 million EU/mL to around 100,000 EU/mL, which is still well above the allowable endotoxin limits for clinical applications and may leave residual Triton X-114 as well. To lower endotoxin levels to below 0.25 EU/mL, the prior art would require a special column chromatography technique, which is very expensive and cannot be used for industrial production.

Current industry practices for effectively removing endotoxins from collagen materials to meet medical standards often involve pretreatment with sodium hydroxide (NaOH). However, the drawback is that NaOH can destroy protein structures, causing denaturation and the inability to achieve optimal protein functionality. In addition, NaOH can also reduce the yield of protein extraction. This problem will be demonstrated later in this application in comparative examples.

Specifically, the processing time and concentration of NaOH used in tissue treatment will affect the tightness, length, and thus denaturation temperature of collagen fibrils. In addition, in the case of industrial production, the excessive use of NaOH will generate a large amount of waste liquid, causing environmental pollution. On the other hand, the chemical agents used in this invention, such as urea and sodium chloride (NaCl, salt), to remove pigments and endotoxins from animal tissues can be treated as general wastewater without causing an environmental burden. Therefore, the tissue treatment process in this invention is harmless to the environment.

Accordingly, developing effective methods to remove endotoxins and melanin from animal tissues to medically approved levels and increase the denaturation temperature of procollagen is a problem to be overcome in the technical field, and this invention does solve this problem. The techniques of this invention do not require the use of NaOH, alcohol, or other chemical liquids that need to be recovered during the process of removing endotoxins and can significantly reduce the processing time and power consumption, providing a novel and green extraction process for mass production.

Therefore, it is one aspect, advantage, objective and embodiment of the present invention to provide a novel and nonobvious method of producing procollagen at industrial scale.

Therefore, it is one aspect, advantage, objective and embodiment of the present invention to provide a method of extracting procollagen from freshwater fish, including parts of the fish not otherwise commercially desirable. It is also one aspect, advantage, objective and embodiment of the present invention to provide a method of extracting procollagen from freshwater fish without using NaOH, alcohol or other pollutants. It is also one aspect, advantage, objective and embodiment of the present invention to provide a method of producing procollagen at significantly lower cost than synthesis.

These and other advantages and aspects of the present invention will be understood from the present application.

### SUMMARY OF THE INVENTION

The present invention teaches that high quality tight twisted triple helix procollagen may be extracted from the skin tissues and organs of freshwater fish. The proper method involves using a sequence of pressure extrusion, mechanical reduction, mixing and emulisification followed by homogenization, refrigeration and cleaning. At the next step an enzymatic extraction process is used followed by multiple rounds of homogenization along with inactivation and finally, filtration.

The result is a low cost, high volume method of producing procollagen with allowably low endotoxin levels (below required limits for clinical applications).

NaOH and alcohol are both not needed for the present invention's process and furthermore, the present invention produces very high yields of the procollagen (up to 80%) compared to processes attempting to use NaOH (yields of 3% or significantly less).

It is therefore one aspect, advantage, objective and embodiment of the invention, in addition to those discussed above, to provide a method of preparing procollagen from freshwater fish, comprising the steps:
i. Pressure treatment: Using high-pressure extrusion to remove blood, water, and fat from freshwater fish tissue containing collagen;
ii. Mechanically separate the freshwater fish tissue; Physical crushing: Cutting and crush the freshwater fish tissue into chunks, strips, or a powder;
iii. Mixing and emulsification: Mixing the freshwater fish tissue particles with a surfactant, an electrolyte such as Na-X (organic salt), and urea to obtain an emulsion solution;
iv. Homogenization: Homogenizing the emulsion solution for 13 to 20 minutes;
v. Refrigeration: Refrigerating the emulsion solution at 0 to 10° C. for 5 to 60 minutes;
vi. Cleaning: Washing the emulsion solution with water at 38 to 42° C. until the absorbance value of the cleaning solution at 200 to 300 nm approaches zero to reduce endotoxins in the freshwater fish tissue to below 0.25 EU/ml;
vii. Extraction: Mixing the freshwater fish tissue with an enzyme and an acid solution of pH 3 to 6 to obtain an extract solution;
viii. Homogenization: Homogenizing the extract solution at 4 to 75 C for 2 to 48 hours;
ix. Inactivation: Adding an enzyme inhibitor to the extract solution to lower the pH to below 3;
x. Homogenization: Homogenizing the inactivated extract solution at 4 to 75° C. for 2 to 48 hours; and
xi. Filtration: Separating the supernatant from the homogenized extract solution through filtration, centrifugation, and/or sieving to obtain the desired procollagen.

It is therefore one aspect, advantage, objective and embodiment of the invention, in addition to those discussed above, to provide a method of preparing procollagen from freshwater fish, wherein said NaCl is added in the amount of between 1 to 5 wt %.

It is therefore one aspect, advantage, objective and embodiment of the invention, in addition to those discussed above, to provide a method of preparing procollagen from freshwater fish, wherein said urea is added in the amount of between 3 to 10 wt %.

It is therefore one aspect, advantage, objective and embodiment of the invention, in addition to those discussed above, to provide a method of preparing procollagen from freshwater fish, wherein said surfactant is selected from the group consisting of Tween 20, Tween 80, Triton X-100, and mixtures thereof, and added in the amount of between 0.05 and 0.5 wt %.

It is therefore one aspect, advantage, objective and embodiment of the invention, in addition to those discussed above, to provide a method of preparing procollagen from freshwater fish, wherein said enzyme activity of enzyme is between 20 and 2000 U, and said enzyme is added in the amount of between 0.5 and 10 wt %.

It is therefore one aspect, advantage, objective and embodiment of the invention, in addition to those discussed above, to provide a method of preparing procollagen from freshwater fish, wherein said enzyme is selected from the group consisting of papain, bromelain, and mixtures thereof.

It is therefore one aspect, advantage, objective and embodiment of the invention, in addition to those discussed above, to provide a method of preparing procollagen from freshwater fish, wherein said acid solution is selected from the group consisting of acetic acid, citric acid, lactic acid, and mixtures thereof, and added in the amount of between 0.5 to 1M.

It is therefore one aspect, advantage, objective and embodiment of the invention, in addition to those discussed above, to provide a method of preparing procollagen from freshwater fish, wherein said enzyme mixtures are a combination of bromelain and papain, and the mixing ratio of bromelain to papain is between 0:1 and 1:2.

It is therefore one aspect, advantage, objective and embodiment of the invention, in addition to those discussed above, to provide a method of preparing procollagen from freshwater fish, wherein said enzyme inhibitor is selected from acetic acid, lactic acid and mixtures thereof, and the mixing ratio of acetic acid to lactic acid is between 1:0 and 1:3.

It is therefore one aspect, advantage, objective and embodiment of the invention, in addition to those discussed above, to provide a method of preparing procollagen from freshwater fish, wherein the electrolyte is an organic salt having the form Na-X.

It is therefore one aspect, advantage, objective and embodiment of the invention, in addition to those discussed above, to provide a method of preparing procollagen from freshwater fish, wherein the mechanical separation of the freshwater fish tissues further comprises cutting and crushing the freshwater fish tissues into one member selected from the group consisting of: powder, chunks, strips and combinations thereof.

It is therefore one aspect, advantage, objective and embodiment of the invention, in addition to those discussed above, to provide a method of preparing procollagen from freshwater fish, wherein the Separation of the supernatant from the homogenized extract solution further comprises one member selected from the group consisting of filtration, centrifuging, sieving and combinations thereof.

### BRIEF DESCRIPTION OF DRAWINGS

FIG. 1 is the chart of the method of this invention for procollagen preparation.

FIGS. 2A(A), 2A(B) and 2A(C) show the macroscopic views of freshwater fish particles before and after the removal of melanin and endotoxins in the embodiments and comparative examples.

FIGS. 2B(A), 2B(B), 2B(C) and 2B(D) show the macroscopic views of the products (supernatant and sediments) of the embodiments and comparative examples following the extraction of procollagen.

FIG. 3A is the Fourier Transform Infrared (FTIR) spectrogram of Embodiment 1 of this invention.

FIG. 3B is the FTIR spectrogram of Comparative Example 1 of this invention.

FIG. 4A is the dynamic light scattering (DLS) spectrogram of Embodiment 1 of this invention.

FIG. 4B is the DLS spectrogram of Comparative Example 1 of this invention.

FIG. 5A is the electropherogram of the sodium dodecyl sulfate-polyacrylamide gel electrophoresis (SDS-PAGE) of all the embodiments of this invention.

FIG. 5B is the SDS-PAGE electropherogram of all the comparative examples #1 and #2 of this invention.

FIG. 6A is a graph of the differential scanning calorimetry (DSC) of Embodiment 1 of this invention.

FIG. 6B is a graph of the DSC of Comparative Example 1 of this invention.

FIG. 7 is an image of the endotoxins test results of procollagen in Embodiment 1 of this invention.

FIG. 8 is the diagram of endothelial stimulation of procollagen in Embodiment 1 of this invention.

FIG. 9 is a chart of the amino acid composition analysis of Embodiment 1 of this invention.

### DETAILED DESCRIPTION OF THE DRAWINGS AND DESCRIPTION OF THE INVENTION

The skin tissue and internal organs of freshwater fish are rich in collagen. As mentioned earlier, an intact collagen structure has a huge effect on promoting the generation and stability of collagen EMC in the human body, thereby protecting the skin. This invention selects freshwater fish as the object, using raw animal tissues as the source of procollagen, applying the techniques of this invention, to obtain in an environmentally friendly manner natural procollagen for medical and commercial use.

This invention aims to solve the problem of prior inadequate techniques used in collagen production by providing an eco-friendly method to effectively remove the endotoxins and melanin in freshwater fish tissue to medically allowable levels without using NaOH, and extract and prepare natural, structurally intact procollagen that is stable in nature and meets the standards for medical use.

This invention provides a method of preparing procollagen from freshwater fish, comprising the steps of:
Pressure treatment: Use high-pressure extrusion to remove blood, water, and fat from freshwater fish tissue containing collagen;
Physical crushing: Cut and crush the freshwater fish tissue into chunks, strips, or a powder;
Mixing and emulsification: Mix the freshwater fish tissue particles with a surfactant, a Na-X such as NaCl, and urea to obtain an emulsion solution;
Homogenization: Homogenize the emulsion solution for 13 to 20 minutes;
Refrigeration: Refrigerate the emulsion solution at 0 to 10° C. for 5 to 60 minutes;
Cleaning: Wash the emulsion solution with water at 38 to 42° C. until the absorbance value of the cleaning solution at 200 to 300 nm approaching zero to obtain the freshwater fish tissue with endotoxins below 0.25 EU/ml;
Extraction: Mix the freshwater fish tissue with an enzyme and an acid solution of pH 3 to 6 to obtain an extract solution;
Homogenization: Homogenize the extract solution at 4 to 75° C. for 2 to 48 hours;
Inactivation: Add an enzyme inhibitor to the extract solution to lower the pH to below 3;
Homogenization: Homogenize the inactivated extract solution at 4 to 75° C. for 2 to 48 hours; and
Filtration: Separate the supernatant from the homogenized extract solution through filtration, centrifugation, and/or sieving to obtain the desired procollagen.

In an embodiment of this invention, NaCl is added in the amount of between 1 and 5 wt %.

In an embodiment of this invention, urea is added in the amount of between 3 and 10 wt %.

In an embodiment of this invention, surfactant is selected from one of the group consisting of Tween 20, Tween 80 and Triton X-100, and mixtures thereof, and added in the amount of between 0.05 and 0.5 wt %.

In an embodiment of this invention, the enzymatic activity of enzyme can range from 20 to 2000 U, and the enzyme is added in the amount of between 0.5 and 10 wt %.

In an embodiment of this invention, enzyme is selected from papain or bromelain or mixtures thereof.

In an embodiment of this invention, the acid solution is selected from the group consisting of acetic acid, citric acid, lactic acid and mixtures thereof, and added in the amount of between 0.5 to 1M.

In an embodiment of this invention, the enzyme mixture is a combination of bromelain and papain, and the mixing ratio of bromelain to papain ranges from 0:1 to 1:2.

In an embodiment of this invention, the enzyme-inhibitor is selected from acetic acid, lactic acid, and mixtures thereof, and the mixing ratio of acetic acid to lactic acid ranges from 1:0 to 1:3.

One of the advantageous effects of this invention is that natural procollagen from freshwater fish is obtained without using the existing technique of NaOH cleaning for animal tissues in raw materials. The method of this invention effectively removes pigments and endotoxins to below 0.25 EU/ml, making the processed freshwater fish tissue clinically applicable by medical standards.

Furthermore, this invention shortens the preparation process and achieves a yield of 60 to 80% for the production of procollagen. The preparation method of procollagen can increase the denaturation temperature of the processed procollagen and prepares procollagen that can be proved safe through cytotoxicity testing and animal intradermal sensitivity testing for use in medical-grade cosmetics, skincare, and medical purposes.

In addition, the method of this invention can extract and retain more natural procollagen components. The procollagen prepared in this invention possesses type I procollagen with structurally intact and tightly twisted triple helices.

To further understand the features and technical components of this invention, please refer to the detailed description and figures provided below. However, the provided figures are for reference and illustration purposes only and are not intended to limit the scope of this invention.

### Implementation, and Test Results Compared to Other Methods

The following are specific embodiments illustrating the method of preparing collagen from freshwater fish according to this invention. Those skilled in the art can understand the advantages and effects of this invention from the description of the implementation. This invention can be implemented or applied through other different embodiments, and various modifications and changes can be made to the details in this implementation based on different perspectives and applications without departing from the concept of this invention. The following embodiments will further describe the relevant techniques of the present invention in detail, but the disclosed content is not intended to limit the scope of protection of the present invention.

It should be noted that although the terms “first,” “second,” “third,” etc. may be used herein to describe various elements and the like, these elements should not be limited by these terms. These terms are only used to distinguish one element from another. Additionally, as used herein, the term “or” may include any and all combinations of one or more of the listed items as applicable depending on the context.

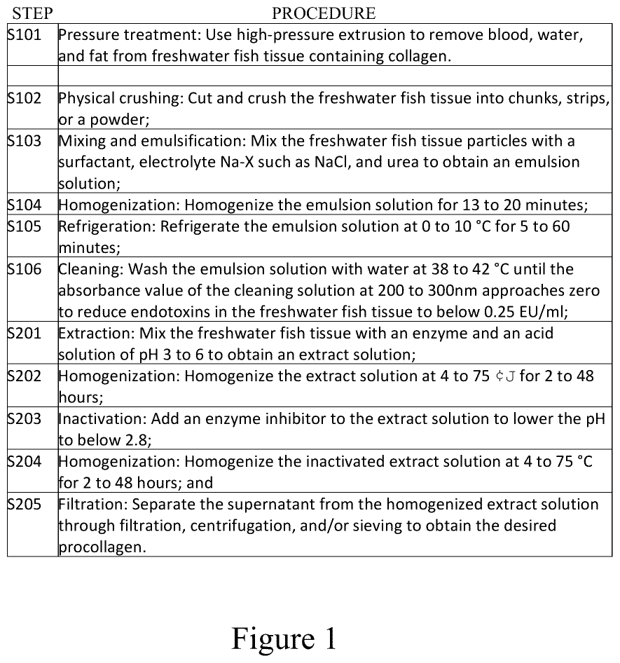

First of all, please refer to FIG. 1. This invention provides a method of preparing procollagen, comprising the following steps: S101 pressure treatment, S102 physical crushing, S103 mixing and emulsification, S104 homogenization, S105 refrigeration, S106 cleaning, S201 extraction, S202 homogenization, S203 inactivation, S204 homogenization, and S205 filtration.

Specifically, steps S101 through S106 are the process of pretreatment used to remove pigments and endotoxins from freshwater fish tissue. Steps S201 through S205 are the process of extraction used to obtain procollagen (collagen) components from freshwater fish tissue. These steps are performed in the order listed below:
S101 Pressure treatment: high-pressure extrusion is used to remove blood, water, and fat from freshwater fish tissue. The freshwater fish tissue can be fish skin or fish organ tissue rich in collagen.
S102 Physical crushing: the said freshwater fish tissue is cut and crushed into chunks, strips, or powder.
S103 Mixing and emulsification: the said freshwater fish tissue is mixed with a surfactant, a Na-X such as NaCl, and urea to obtain an emulsion solution.

Preferably, the surfactant is a stock solution selected from Tween 20, Tween 80, and Triton X-100, although combinations thereof may be employed. The surfactant is added in the amount of between 0.05 and 0.5 wt %. Preferably, organic salts are used for the NaCl. The NaCl is added or other Na-X can be used in the amount of between 1 to 5 wt %. The urea is added in the amount of between 3 to 10 wt %.

The reason why NaCl can reduce endotoxins is that it has the function of regulating osmotic pressure. NaCl increases the osmotic pressure in the extracellular fluid, which can cause a pressure difference between the extracellular fluid and intracellular fluid, thereby inhibiting the release of endotoxins by bacteria. This is because high osmotic pressure in the extracellular fluid causes loss of intracellular water, leading to cell shrinkage, thus reducing cell death and the release of endotoxins.

In addition, NaCl can further reduce the release of endotoxins by inhibiting the breakdown of the cell wall and the release of proteases. The osmotic pressure regulation function of NaCl can reduce the release of endotoxins by bacteria, making a positive impact on the body's immune system.

Furthermore, other organic salts such as Na-X can reduce the release of endotoxins by promoting the stability of membrane lipids and the strength of the cell wall.
S104 Homogenization: Homogenize the emulsion solution using ultrasound, stirring, and shaking methods for 13 to 20 minutes.
S105 Refrigeration: Refrigerate the emulsion solution at 0 to 10° C. for 5 to 60 minutes. Ideally, the refrigeration temperature should be 4° C.
S106 Cleaning: Wash the emulsion solution with water at 38 to 42° C. until the absorbance value of the cleaning solution at 200 to 300 nm approaches zero to reduce the endotoxins to below 0.25 EU/ml in the freshwater fish tissue.
S201 Extraction: Mix the freshwater fish tissue with an endotoxin level of below 0.25 EU/ml with an enzyme and an acidic solution of pH 3 to 6 to obtain an extraction solution.

Specifically, the enzymatic activity of the enzyme can range from 20 to 2000 U, preferably 2000 U. Furthermore, the enzyme may be selected from the group consisting of papain, bromelain, and mixtures thereof, and added in the amount of between 0.5 to 10 wt %. The acid solution may be selected from the group consisting of acetic acid, citric acid, lactic acid, and mixtures thereof, and added in the amount of between 0.5 to 1M.

Preferably, the enzyme solution is a mixture of bromelain and papain. The mixing ratio of bromelain to papain ranges from 0:1 to 1:2.
S202 Homogenization: Homogenize the extract solution using ultrasound, stirring, and shaking methods at 4 to 75° C. for 2 to 48 hours.
S203 Inactivation: Lower the pH of the extract solution to below pH 3 using an enzyme inhibitor, preferably below 2.8.

The enzyme inhibitor is selected from acetic acid and lactic acid, preferably a mixture of both. The mixing ratio of acetic acid to lactic acid ranges from 1:0 to 1:3.
S204 Homogenization: Homogenize the inactivated extract solution using ultrasound, stirring, and shaking methods at 4 to 75° C. for 2 to 48 hours.
S205 Filtration: Collect the supernatant through filtration, centrifugation, and/or sieving of the extract solution to obtain the desired procollagen.

Use the implementation steps described above to implement the three embodiments of this invention using the concentrations and ratios provided below in the formula composition tables for the embodiments.

### Embodiment 1

The formula composition of Embodiment 1 is shown in Table 1.

TABLE 1
Embodiment 1

| | Surfactant | NaCl | Urea | Acid | enzyme | Extraction |
|---|---|---|---|---|---|---|
| NaOH | (wt %) | (wt %) | (wt %) | (M) | (wt %) | time (hr) |
| — | Tween 80: | 1 wt % | 5 wt % | Acetic acid:lactic | bromelain:papain | 8 hrs |
| | 0.05 wt % | | | acid 1:1.5 | 1:2 | |
| | | | | Adjust the pH of the | | |
| | | | | acid solution to 4 | | |
| | | | | for extraction | | |

### Embodiment 2

The formula composition of Embodiment 2 is shown in Table 2

TABLE 2
Embodiment 2

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| — | — | 5 wt % | 10 wt % | Acetic acid:lactic | Bromelain:Papain | 8 hrs |
| | | | | acid 1:1 | 1:1 | |
| | | | | Adjust the pH of the | | |
| | | | | acid solution to 4 | | |
| | | | | for extraction | | |

### Embodiment 3

The formula composition of embodiment 3 is as shown in table 3

TABLE 3
Embodiment 3

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| — | Tween 80 | 1 wt % | 3 wt % | Acetic acid; | Bromelain:Papain | 8 hrs |
| | 0.05 wt % | | | Adjust the pH of the | 0:1 | |
| | | | | acid solution to 4 | | |
| | | | | for extraction | | |

### Comparative Examples

A fragment of freshwater fish tissue was prepared and subjected to high-pressure extrusion to remove blood, water, and fat. The fragment was then broken into chunks, strips, or a powder through physical cutting and crushing. Next, the particles of the freshwater fish tissue were washed with a NaOH solution until the endotoxins in the freshwater fish tissue were reduced to below 0.25 EU/ml.

In Comparative Example 1, the particles of freshwater fish tissue were cleaned with NaOH at 4° C. for 24 hours; in Comparative Example 2, the particles of freshwater fish tissue were cleaned with NaOH at 4° C. for 30 hours.

The particles cleaned with NaOH are then mixed with 0.8 M acetic acid and 0.1 wt % porcine pepsin to obtain an extract solution. The extract solution was homogenized at 4° C. and then extracted for 240 hours. The supernatant that was formed following the extraction was then collected through filtration, centrifugation, and/or sieving to obtain the desired procollagen of comparative examples.

### Comparative Example 1

The formula composition of comparative example 1 is as shown in table 4.

TABLE 4
comparative example 1

| | surfactant | NaCl | Urea | Acid | Enzyme | Extraction |
|---|---|---|---|---|---|---|
| NaOH | (wt %) | (wt %) | (wt %) | (M) | (wt %) | time (hr) |
| 0.1M | — | — | — | Acetic | Porcine | 240 hrs |
| | | | | acid | pepsin | |
| | | | | 0.8M | 0.1 wt % | |

### Comparative Example 2

Comparative Example 2 was conducted using the implementation steps described in Comparative Example 1, and its formulation was adjusted as shown in Table 5.

TABLE 5
comparative example 2

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| 0.5M | — | — | — | Acetic | Porcine | 240 hrs |
| | | | | acid | pepsin | |
| | | | | 0.8M | 0.1 wt % | |

### Observation of Pigment Removal Effect

Pretreatments of freshwater fish tissue fragments for endotoxin removal were conducted in all of the embodiments and comparative examples, and observation and a comparison of an untreated fragment and the pretreated fragments were performed with the naked eye. The untreated fragment and the pretreated fragments from the comparative examples appeared light grey, whereas the fragments from the embodiments clearly appeared off-white, indicating that the embodiments are more effective than the comparative example 1 in removing melanin.

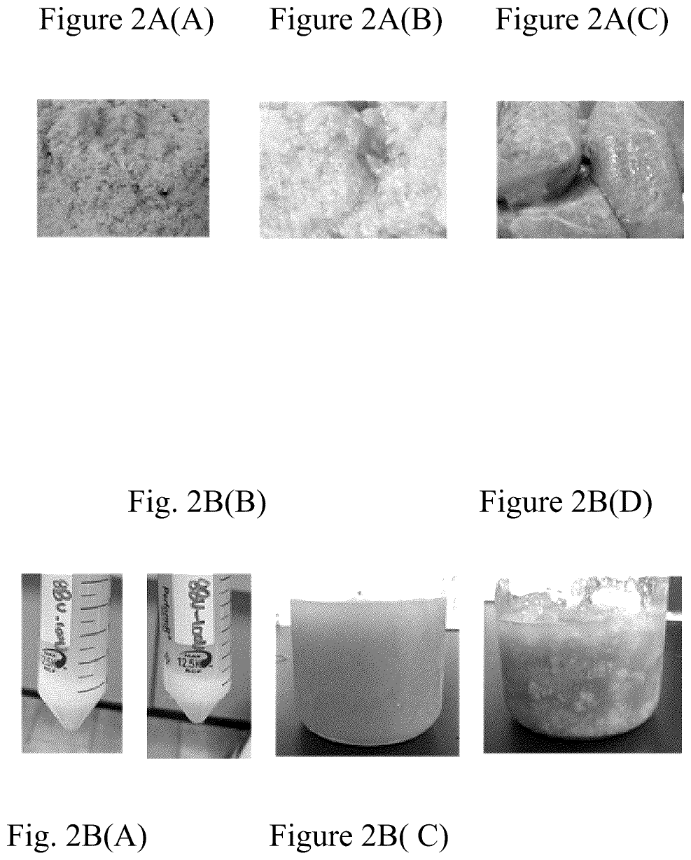

See FIG. 2A. FIG. 2A shows the macroscopic views of:
(A) Organ particles of freshwater fish containing collagen before pretreatment (Sample A);
(B) Organ particles of freshwater fish containing collagen after being pretreated with a surfactant, urea, and NaCl, and then washed with clean water (Sample B); and
(C) Particles of freshwater fish containing collagen after being pretreated with NaOH.

FIG. 2B shows the macroscopic views of:
(A) Sample B after the extraction process with papain and acetic acid;
(B) Sample B with after the extraction process with a mixture of bromelain and papain, then a mixture of lactic acid and acetic acid, and then centrifuged, to get the supernatant and sediments; and
(C) & (D) freshwater fish skin powder after being pretreated with NaOH and then mixed with acetic acid and porcine pepsin, to get supernatant (C) and sediments (D).

### Fourier Transform Infrared (FTIR) Spectroscopy

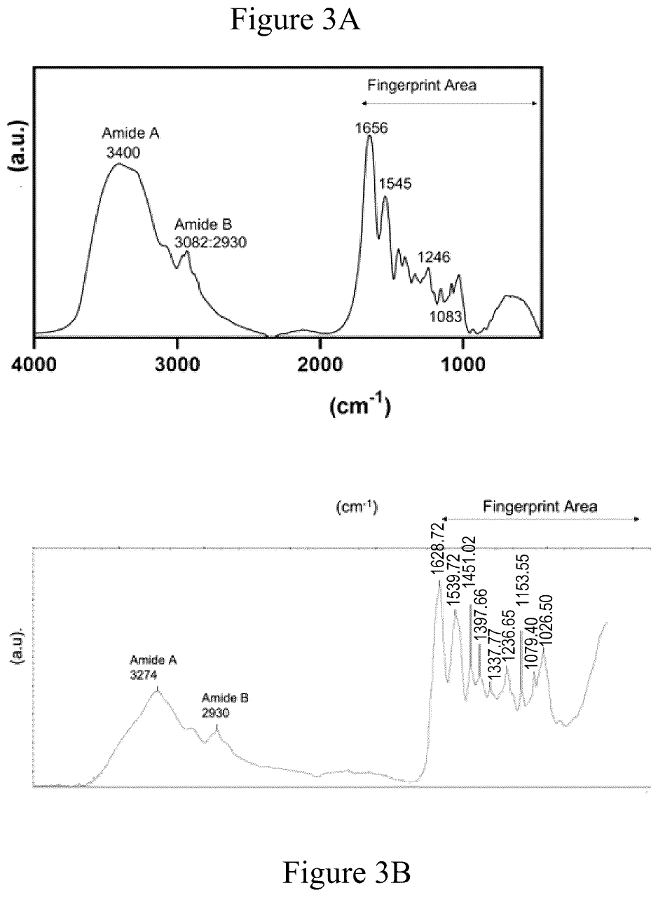

FIG. 3A is the FTIR spectrogram of procollagen in Sample B from the embodiments of this invention after an extraction with a mixture of papain and bromelain and a mixture of acetic acid and lactic acid.

As shown in FIG. 3A, Fourier transform infrared (FTIR) spectroscopy can distinguish several characteristic absorption bands of collagen: amide I at around 1656 cm-1, amide II at around 1540 cm-1, and a set of three weaker bands representing the amide III vibration mode centered at around 1246 cm-1.

The amide I band arises from the stretching and bending vibrations of the peptide carbonyl group (—CO). A peak at 1083 cm-1 appears in the spectrum of fibrous collagen and is slightly shifted from the peaks of amide I and II. Therefore, by comparing the spectra in FIG. 3A, we can identify a specific feature appearing as a peak at 1083 cm-1, which allows us to recognize that the embodiments possess the structure of procollagen.

FIG. 3B is a FTIR spectrogram of procollagen from a comparative example of this invention. It can be seen from the figure that there is a peak at 3274 cm-1, which is Amide A. It is found that compared with FIG. 3A, there is a shift in the peak position, and the peak height of Amide A is lower than that in FIG. 3A, indicating that the alpha helix structure in the comparative example is less stable and has a lower content than that in the embodiments. The same situation is observed for Amide B. In addition, there is a small peak at 1080 cm-1, which represents only a small amount of procollagen.

### Animal Intracutaneous Irritation Test

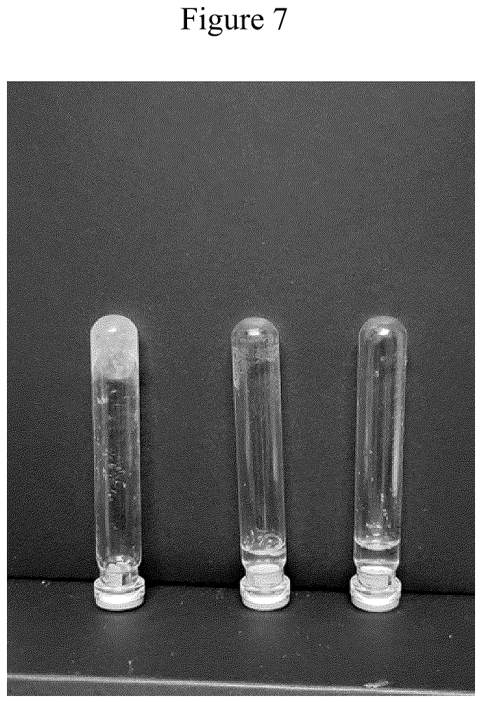

An animal intracutaneous irritation test was conducted according to ISO10993-1 to evaluate the safety of the extracted procollagen. In the test, both polar and nonpolar collagen extracts were injected intradermally in animals to observe any signs of redness, swelling, or heat. After 72 hours, no such symptoms were observed, and the score was between 0 and 1. Therefore, the embodiments showed no intracutaneous irritation. See FIG. 8. FIG. 8 is an analysis of the endothelial stimulation (according to ISO10993-1) of procollagen from an embodiment of this invention. The data of the endothelial stimulation were collected 72 hours after the subcutaneous injections into New Zealand white rabbits of procollagen that was extracted using a mixture of papain and bromelain and a mixture of acetic acid and lactic acid.

Polar refers to the procollagen extracted with physiological saline for 24 hours and injected subcutaneously into New Zealand white rabbits. Polar control group (Polar Control) refers to physiological saline. Non-polar refers to the procollagen extracted with cottonseed oil for 24 hours and injected subcutaneously into New Zealand white rabbits. Non-polar control group (Non-Polar Control) refers to the cottonseed oil.

### Triple Helix Characteristic Test

The ultraviolet-visible (UV-VIS) absorption spectrum of a protein is mainly determined by the peptide bonds or side chains of the protein. The amino acid sequence of the protein contains glycine, proline, and hydroxyproline, and the triple-helix collagen has a maximum peak at about 240 nm.

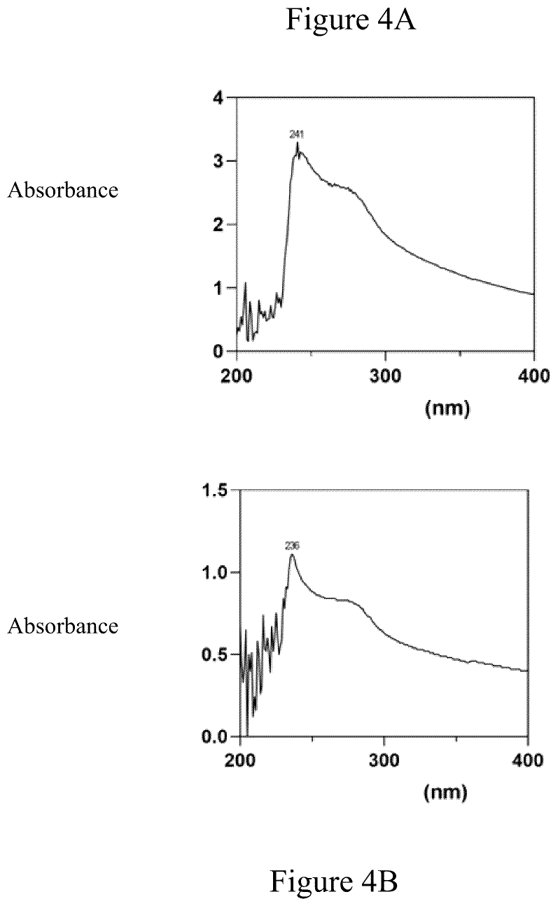

FIG. 4A is the dynamic light scattering (DLS) spectrogram of procollagen in Sample B after an extraction with a mixture of bromelain and papain and a mixture of lactic acid and acetic acid. As shown in FIG. 4A, dynamic light scattering (DLS) was used to detect the full spectrum of Embodiment 1. Embodiment 1 showed the maximum absorbance at around 240 nm, with no peaks at other wavelengths, indicating that the triple helix of Embodiment 1 is tightly twisted, and no collagen fragments were generated, demonstrating that Embodiment 1 possesses intact type I procollagen.

FIG. 4B is the DLS spectrogram of procollagen from comparative examples. As shown in FIG. 4B, the full spectrum of Comparative Example 1 was detected using DLS. Comparative Example 1 showed the maximum absorbance at around 240 nm, but the absorbance was not as high as that of Embodiment 1, indicating that the amount of triple helical collagen in Comparative Example 1 was lower than that in Embodiment 1. Moreover, Comparative Example 1 also showed absorbance peak signals between 200 and 240 nm, indicating that Comparative Example 1 had many short-wavelength collagen fragments. In other words, compared to Embodiment 1, the purity of triple-helical collagen obtained from Comparative Example 1 was lower.

### Sodium Dodecyl Sulfate Polyacrylamide Gel Electrophoresis

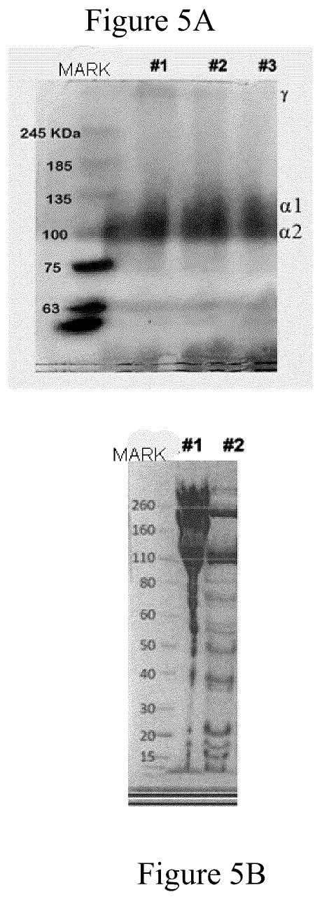

FIG. 5A shows the electropherogram of sodium dodecyl sulfate polyacrylamide gel electrophoresis (SDS-PAGE) of procollagen of #1 embodiments 1, #2 embodiments 2, and embodiments 3.

As shown in FIG. 5A, the result of sodium dodecyl sulfate polyacrylamide gel electrophoresis (SDS-PAGE) shows that Embodiment 1 has α1 and α2 chains, indicating that it is type I procollagen. In addition, the y-chain (trimer) also shows the presence of high molecular weight cross-linking.

FIG. 5B shows the SDS-PAGE electropherogram of skin particles of freshwater fish:
(#1) after a pretreatment with NaOH for 24 hours and then and extraction with a mixture of acetic acid and porcine pepsin for 240 hours.
(#2) after a pretreatment with NaOH for 30 hours and then an extraction with a mixture of acetic acid and porcine pepsin for 240 hours.

As shown in FIG. 5B, for both Comparative Example 1 treated with NaOH for 24 hours and Comparative Example 2 treated for 30 hours, not only α1 and α2 chains and y chains were shown but so was a low molecular weight sequence fragment. Compared to Embodiment 1, there were many low molecular weight sequence fragments in the collagen of Comparative Examples 1 and 2, indicating that the triple-helix structure of collagen in Comparative Example 1 was not able to maintain its characteristics and was easily degraded into small fragments.

### Cell Cytotoxicity Test

The cell cytotoxicity test was conducted on mouse fibroblast cells (L929) in vitro to evaluate the effect of Embodiment 1 on the survival rate of L929. Cell survival rate was assessed using MTT assay analysis. According to the ISO10993-1 method for cell cytotoxicity, after treatments for 24, 48, and 72 hours, the survival rates were approximately 75%, 82%, and 88%, respectively, indicating that the procollagen obtained from Embodiment 1 does not exhibit cell toxicity.

### Denaturation Temperature

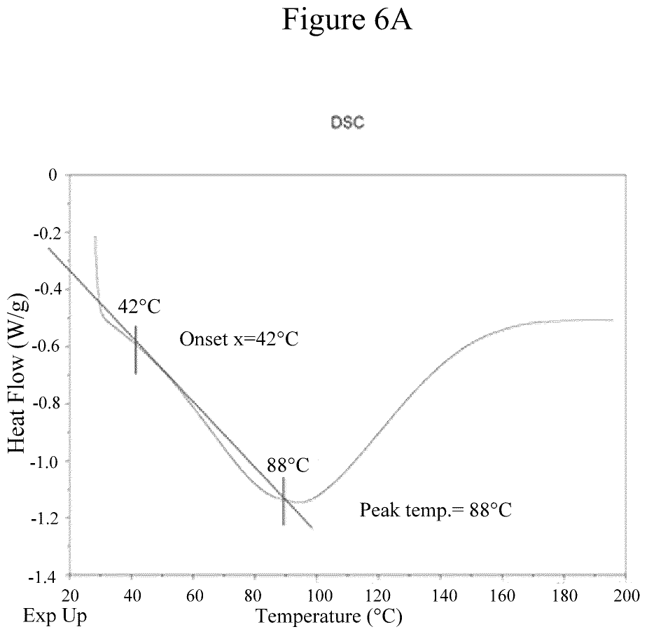

The denaturation temperature was determined using differential scanning calorimetry, and the test results are recorded in Table 6 and FIGS. 6A and 6B. FIG. 6A is the graph of differential scanning calorimetry (DSC) of procollagen in an embodiment of this invention after an extraction with a mixture of papain and bromelain and a mixture of acetic acid and lactic acid. FIG. 6B is a graph of DSC of procollagen in comparative examples.

As shown in FIG. 6A, the denaturation temperatures of the embodiments were approximately between 77 and 88° C., whereas the denaturation temperatures of the comparative examples (see FIG. 6B) were approximately 45° C. Therefore, it can be concluded that Embodiment 1 has a more tightly packed/twisted triple-helix structure.

### Endotoxin Content Detection

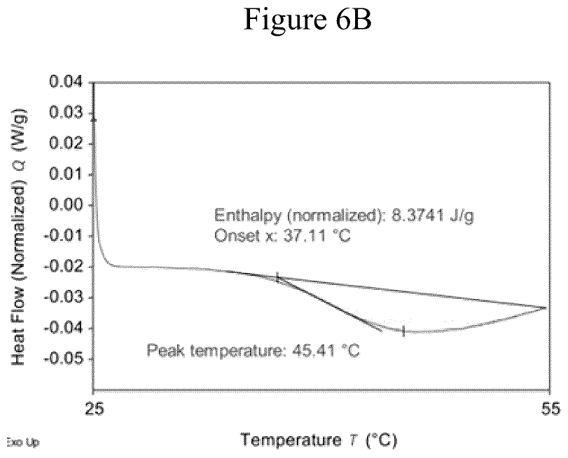

The endotoxin content was determined using the GEL-CLOT LAL assay. The LAL reagent used in the test forms a gel clot when reacting with endotoxin. The formation of the gel is observed with the naked eye to detect the presence of endotoxin. The test results for the endotoxin content are shown in Table 6 and FIG. 7. FIG. 7 is an image of the endotoxins test results of procollagen in an embodiment of this invention. (Gel-Clot test)
(A) Positive control group, with endotoxins greater than 0.25 EU/ml
(B) Procollagen in Sample B from an embodiment after an extraction with a mixture of papain and bromelain and a mixture of acetic acid and lactic acid
(C) Blank group: this sample is endotoxin-free water

### Protein Analysis

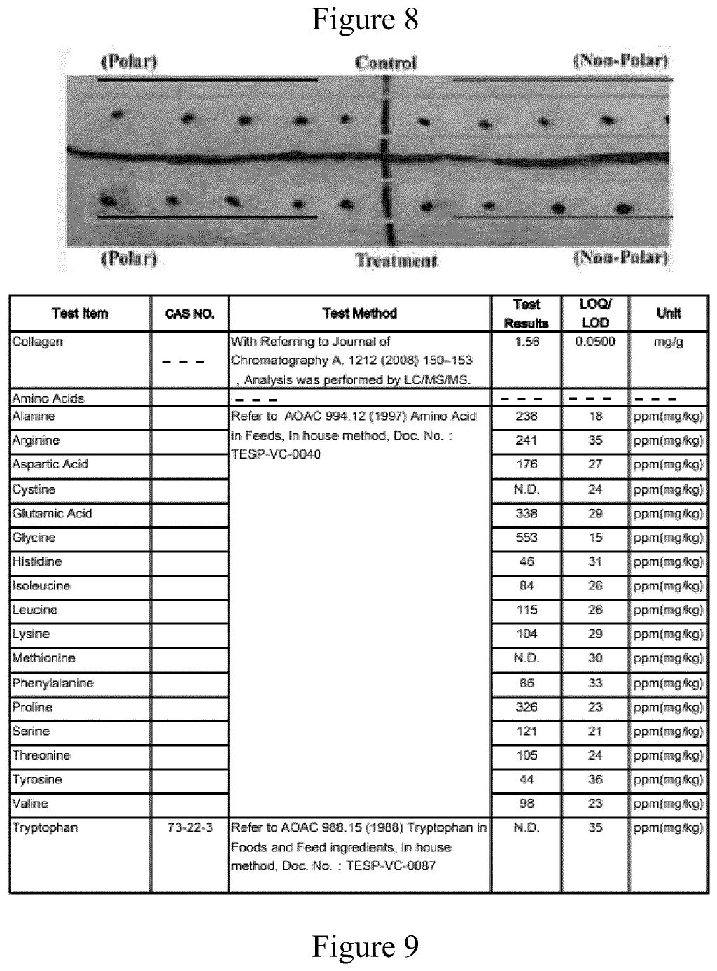

The protein concentration and amino acid sequence were analyzed using LC-MS/MS. FIG. 9 shows the analysis of the amino acid composition of procollagen in Sample B from the embodiments after an extraction with a mixture of papain and bromelain and a mixture of acetic acid and lactic acid. Amino acid composition analysis of collagen from comparative examples results are recorded in Table 6. Embodiments 1 through 3 had high protein concentrations of 1 mg/g to 1.56 mg/g, while Comparative Example 1 had a protein concentration of only 0.2 mg/g. In addition, proline, glycine, glutamine, and arginine are precursors of procollagen, and the higher content they are, the tighter and more concentrated the triple-helix structure of procollagen will be. As shown in Table 6, Embodiment 1 had a much higher amino acid content compared to Comparative Example 1.

TABLE 6

| | | denaturation | | Protein | *Proline/Glycine/ |
|---|---|---|---|---|---|
| | | temperature | | concentration | Glutamine/Arginine |
| | yield | (° C.) | Endotoxin (EU/ml) | (mg/g) | content (ppm) |
| Embodiment 1 | 55-80% | 88 | Non-gelling state <0.25 | 1.56 | 326/553/338/241 |
| Embodiment 2 | 45-60% | 77 | Non-gelling state <0.25 | 1.5 | — |
| Embodiment 3 | 30-45% | — | Non-gelling state <0.25 | 1 | — |
| Comparative example 1 | 2-3% | 45 | Non-gelling state <0.25 | 0.2 | 127/374/75/58 |
| Comparative example 2 | 0.5-1% | 45 | Non-gelling state <0.25 | | |

### Advantageous Effects of Embodiments

One of the advantageous effects of this invention is that the method of the invention replaces the existing technique of using NaOH cleaning while effectively removes pigments and endotoxins from freshwater fish tissue to below 0.25 EU/ml, making the treated freshwater fish tissue clinically applicable by medical standards.

Furthermore, this invention shortens the preparation process and achieves a yield of over 80% for the production of procollagen. The preparation method of procollagen in this invention can increase the denaturation temperature of the pretreated procollagen. Through cytotoxicity testing and animal skin sensitivity testing, it can be safely used in skin care, cosmetic, and medical products.

By using the preparation method of collagen provided by this invention, more procollagen components can be extracted and retained, and the prepared procollagen possesses structurally intact and tightly twisted triple-helix type I procollagen.

Another advantage of the method of this invention is its achievement of the goal of maintaining a green environment. All the materials used in this invention are “food-grade” materials, and the wastewater discharged during the preparation process is general wastewater, which does not cause environmental pollution or burden. The important aspect of the method of this invention is to achieve high yields of quality procollagen with endotoxins below 0.25 EU/ml while also taking into account protection of the environment.

This invention can be implemented in large-scale industrial production to produce procollagen that provides high value to the commercial and medical fields, promoting the development of biotechnology.

The embodiments disclosed above are only the preferred ones of this invention and do not limit the scope of the patent application. Therefore, any equivalent technical changes made using the implementation and figures of this invention are included within the scope of the patent application of this invention.

The disclosure is provided to render practicable the invention by those skilled in the art without undue experimentation, including the best mode presently contemplated and the presently preferred embodiment. Nothing in this disclosure is to be taken to limit the scope of the invention, which is susceptible to numerous alterations, equivalents and substitutions without departing from the scope and spirit of the invention. The scope of the invention is to be understood from the appended claims.

Methods and components are described herein. However, methods and components similar or equivalent to those described herein can be also used to obtain variations of the present invention. The materials, articles, components, methods, and examples are illustrative only and not intended to be limiting.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art.

Having illustrated and described the principles of the invention in exemplary embodiments, it should be apparent to those skilled in the art that the described examples are illustrative embodiments and can be modified in arrangement and detail without departing from such principles. Techniques from any of the examples can be incorporated into one or more of any of the other examples. It is intended that the specification and examples be considered as exemplary only, with a true scope and spirit of the invention being indicated by the following claims.

1. A method of preparing procollagen having a structurally intact triple helix and a denaturation temperature of 86 degrees C. from freshwater fish, comprising the steps:
i. Using pressure extrusion to remove blood, water, and fat from freshwater fish tissue containing collagen;
ii. Mechanically separate the freshwater fish tissue;
iii. Mix the freshwater fish tissue with a surfactant, an electrolyte, and urea to obtain an emulsion solution;
iv. Homogenize the emulsion solution for 13 to 20 minutes;
v. Refrigerate the emulsion solution at 0 to 10° C. for 5 to 60 minutes;
vi. Wash the emulsion solution with water at 38 to 42° C. until the absorbance value of the mixture of water and emulsion solution at 200 to 300 nm approaches zero to reduce endotoxins in the freshwater fish tissue to below 0.25 EU/ml;
vii. Mix the freshwater fish tissue with an enzyme and an acid solution of pH 3 to to obtain an extract solution;
viii. Homogenize the extract solution at 4 to 75° C. for 2 to 48 hours;
ix. Add an enzyme inhibitor to the extract solution to lower the pH to below 3;
x. Homogenize the inactivated extract solution at 4 to 75° C. for 2 to 48 hours; and
xi. Separate the supernatant from the homogenized extract solution to obtain procollagen having a structurally intact triple helix and a denaturation temperature of 86 degrees C.

2. The method of preparing procollagen from freshwater fish of claim 1, wherein the electrolyte is NaCl and the NaCl is added in the amount of between 1 to 5 wt %.

3. The method of preparing procollagen from freshwater fish of claim 1, wherein said urea is added in the amount of between 3 to 10 wt %.

4. (canceled)

5. The method of preparing procollagen from freshwater fish of claim 1, wherein the enzyme has an activity which is between 20 and 2000 U, and said enzyme is added in the amount of between 0.5 and 10 wt %.

6. The method of preparing procollagen from freshwater fish of claim 1, wherein said enzyme is selected from the group consisting of papain, bromelain, and mixtures thereof.

7. The method of preparing procollagen from freshwater fish of claim 1, wherein said acid solution is selected from acetic acid, citric acid, lactic acid, and mixtures thereof, and added in the amount of between 0.5 to 1M.

8. The method of preparing procollagen from freshwater fish of claim 6, wherein said enzyme mixtures are a combination of bromelain and papain, and the mixing ratio of bromelain to papain is between 0:1 and 1:2.

9. The method of preparing procollagen from freshwater fish of claim 1, wherein said enzyme inhibitor is selected from the group consisting of acetic acid, lactic acid and mixtures thereof, and the mixing ratio of acetic acid to lactic acid is between 1:0 and 1:3.

10. The method of preparing procollagen from freshwater fish of claim 1, wherein the electrolyte is an organic salt having the form Na—X.

11. The method of preparing procollagen from freshwater fish of claim 1, wherein the mechanical separation of the freshwater fish tissues further comprises cutting and crushing the freshwater fish tissues into one member selected from the group consisting of: powder, chunks, strips and combinations thereof.

12. The method of preparing procollagen from freshwater fish of claim 1, wherein the separation of the supernatant from the homogenized extract solution further comprises one member selected from the group consisting of filtration, centrifuging, sieving and combinations thereof.
